(19) United States
(12) Patent Application Publication
(10) Pub. No.: US 2025/0366986 A1
(43) Pub. Date: Dec. 4, 2025

(54) REPLACEMENT HEART VALVE IMPLANT SYSTEM

(71) Applicant: BOSTON SCIENTIFIC SCIMED, INC., MAPLE GROVE, MN (US)

(72) Inventors: James Holmberg, Champlin, MN (US); Levi Joel Wolterstorff, Saint Paul, MN (US); James M. Anderson, Corcoran, MN (US); Chenhao Fu, Plymouth, MN (US); Kelsey Rae Cooper, Andover, MN (US); Dennis A Boismier, Shorewood, MN (US); Donal Kyne, Galway (IE); Brandon Campbell, Galway (IE)

(73) Assignee: BOSTON SCIENTIFIC SCIMED, INC., MAPLE GROVE, MN (US)

(21) Appl. No.: 19/226,778

(22) Filed: Jun. 3, 2025

Related U.S. Application Data
(60) Provisional application No. 63/655,662, filed on Jun. 4, 2024.

(51) Int. Cl. A61F 2/24

(52) U.S. Cl. CPC A61F 2/2418; A61F 2/2436; A61F 2210/00; A61F 2220/0025

(57) ABSTRACT

A replacement heart valve system includes a replacement heart valve implant including an expandable framework and a plurality of leaflets, wherein the framework includes an attachment tab extending upstream from an inflow end, and an implant delivery system including an implant holding portion including a stent holder configured to engage the attachment tab to axially secure the implant relative to the delivery system. The stent holder includes a first radiopaque marker, and the attachment tab includes a second radiopaque marker, the first and second markers may collectively indicate a rotational orientation of the framework relative to a native heart valve under fluoroscopy. The first radiopaque marker may indicate when a middle commissure post of the framework is disposed in a posterior position and an anterior position relative to a native heart valve under fluoroscopy in a 3-cusp view.

### CROSS-REFERENCE TO RELATED APPLICATION

This application is a continuation of U.S. Patent Application Ser. No. 63/655,662, filed Jun. 4, 2024, entitled “REPLACEMENT HEART VALVE IMPLANT SYSTEM”, which is incorporated by reference herein in its entirety.

### TECHNICAL FIELD

The disclosure relates generally to medical devices and more particularly to a replacement heart valve implant and/or features for coupling a replacement heart valve implant to an implant delivery system.

### BACKGROUND

A wide variety of intracorporeal medical devices have been developed for medical use including replacement heart valve implants for repair or replacement of diseased heart valves. Of the known medical devices and methods, each has certain advantages and disadvantages. There is an ongoing need to provide alternative medical devices as well as alternative methods for manufacturing and using medical devices.

### SUMMARY

In one example, a replacement heart valve system may comprise a replacement heart valve implant comprising an expandable framework configured to shift between a radially collapsed configuration and a radially expanded configuration, and a plurality of valve leaflets secured to the expandable framework, wherein the expandable framework comprises a tubular wall defining an inflow end and an attachment tab extending upstream from the inflow end, and an implant delivery system comprising a handle and an elongate shaft assembly extending distally from the handle, wherein a distal portion of the elongate shaft assembly comprises an implant holding portion including a stent holder configured to engage the attachment tab in the radially collapsed configuration to axially secure the replacement heart valve implant relative to the implant delivery system. The stent holder may comprise a first radiopaque marker and the attachment tab may comprise a second radiopaque marker, the first radiopaque marker and the second radiopaque marker collectively indicating a rotational orientation of the expandable framework relative to a native heart valve under fluoroscopy.

In addition, or alternatively, to any example disclosed herein, the stent holder is substantially transparent under fluoroscopy.

In addition, or alternatively, to any example disclosed herein, the stent holder comprises a notch configured to receive the attachment tab in the radially collapsed configuration.

In addition, or alternatively, to any example disclosed herein, the first radiopaque marker is disposed on or within a shoulder of the notch.

In addition, or alternatively, to any example disclosed herein, the first radiopaque marker extends in a longitudinal direction.

In addition, or alternatively, to any example disclosed herein, the first radiopaque marker and the second radiopaque marker collectively form a symbol visible under fluoroscopy.

In addition, or alternatively, to any example disclosed herein, the symbol has a first appearance under fluoroscopy when the expandable framework is in a correct rotational orientation relative to the native heart valve, and a second appearance under fluoroscopy when the expandable framework is in an incorrect rotational orientation relative to the native heart valve.

In addition, or alternatively, to any example disclosed herein, the second appearance is a mirror image of the first appearance.

In addition, or alternatively, to any example disclosed herein, the symbol comprises a longitudinal component and a lateral component.

In addition, or alternatively, to any example disclosed herein, under fluoroscopy the lateral component extends in a first direction from the longitudinal component when the expandable framework is in a correct rotational orientation relative to the native heart valve, and the lateral component extends in a second direction opposite the first direction from the longitudinal component when the expandable framework is in an incorrect rotational orientation relative to the native heart valve.

In addition, or alternatively, to any example disclosed herein, and in a second example, a replacement heart valve system may comprise a replacement heart valve implant comprising an expandable framework configured to shift between a radially collapsed configuration and a radially expanded configuration, and a plurality of valve leaflets secured to the expandable framework, wherein the expandable framework comprises a tubular wall defining an inflow end and an attachment tab spaced apart upstream from the inflow end by a neck extending therebetween, and an implant delivery system comprising a handle and an elongate shaft assembly extending distally from the handle, wherein a distal portion of the elongate shaft assembly comprises an implant holding portion including a stent holder configured to engage the attachment tab in the radially collapsed configuration to axially secure the replacement heart valve implant relative to the implant delivery system. The stent holder may comprise a circumferential extension extending radially outward from a body of the stent holder. The circumferential extension may comprise a notch having a proximal aperture configured to receive the neck and a distally facing recess configured to receive the attachment tab. The stent holder may comprise a first radiopaque marker and the attachment tab may comprise a second radiopaque marker, the first radiopaque marker and the second radiopaque marker collectively indicating a rotational orientation of the expandable framework relative to a native heart valve under fluoroscopy.

In addition, or alternatively, to any example disclosed herein, the proximal aperture and the distally facing recess both open radially outward from the circumferential extension.

In addition, or alternatively, to any example disclosed herein, under fluoroscopy a first relative positioning of the first radiopaque marker and the second radiopaque marker collectively indicates a correct rotational orientation of the expandable framework relative to the native heart valve, and a second relative positioning of the first radiopaque marker and the second radiopaque marker collectively indicates an incorrect rotational orientation of the expandable framework relative to the native heart valve.

In addition, or alternatively, to any example disclosed herein, the implant holding portion comprises a distal sheath and a proximal sheath configured to constrain the replacement heart valve implant in the radially collapsed configuration with the attachment tab engaged with the stent holder.

In addition, or alternatively, to any example disclosed herein, and in a third example, a replacement heart valve system may comprise a replacement heart valve implant comprising an expandable framework configured to shift between a radially collapsed configuration and a radially expanded configuration, and a plurality of valve leaflets secured to the expandable framework, wherein the expandable framework comprises a tubular wall defining an inflow end and an attachment tab extending upstream from the inflow end, and an implant delivery system comprising a handle and an elongate shaft assembly extending distally from the handle, wherein a distal portion of the elongate shaft assembly comprises an implant holding portion including a stent holder configured to engage the attachment tab in the radially collapsed configuration to axially secure the replacement heart valve implant relative to the implant delivery system. The stent holder may be substantially transparent under fluoroscopy. The stent holder may comprise a first radiopaque marker embedded therein, the first radiopaque marker indicating when a middle commissure post of the expandable framework is disposed in a posterior position and when the middle commissure post of the expandable framework is disposed in an anterior position relative to a native heart valve under fluoroscopy in a 3-cusp view.

In addition, or alternatively, to any example disclosed herein, the first radiopaque marker has a first appearance under fluoroscopy in the 3-cusp view when the middle commissure post of the expandable framework is disposed in the posterior position, and a second appearance under fluoroscopy in the 3-cusp view when the middle commissure post of the expandable framework is disposed in the anterior position.

In addition, or alternatively, to any example disclosed herein, the second appearance is a mirror image of the first appearance.

In addition, or alternatively, to any example disclosed herein, the first radiopaque marker comprises a longitudinal component and a lateral component.

In addition, or alternatively, to any example disclosed herein, under fluoroscopy in the 3-cusp view, the lateral component extends in a first direction from the longitudinal component when the middle commissure post of the expandable framework is disposed in the posterior position, and the lateral component extends in a second direction opposite the first direction from the longitudinal component when the middle commissure post of the expandable framework is disposed in the anterior position.

In addition, or alternatively, to any example disclosed herein, the stent holder may comprise a circumferential extension extending radially outward from a body of the stent holder, the circumferential extension comprising a notch configured to receive the attachment tab in the radially collapsed configuration;

In addition, or alternatively, to any example disclosed herein, the first radiopaque marker is embedded within the body of the stent holder radially inward from and circumferentially aligned with the notch.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of an implant delivery system for delivering a replacement heart valve implant;

FIG. 3 illustrates selected aspects of a portion of the implant delivery system of FIG. 2 according to the disclosure;

FIG. 4 illustrates selected aspects of a portion of the implant delivery system of FIG. 2 according to the disclosure;

FIGS. 5-8 illustrate selected aspects of a stent holder of the implant delivery system of FIG. 3 engaging a replacement heart valve implant, where FIG. 7 is rotated 180 degrees from FIG. 5;

FIGS. 9-10 illustrate selected aspects of a stent holder of the implant delivery system of FIG. 3 engaging a replacement heart valve implant, where FIG. 10 is rotated 180 degrees from FIG. 9;

FIGS. 11-12 illustrate selected aspects of a stent holder of the implant delivery system of FIG. 3, where FIG. 12 is rotated 180 degrees from FIG. 11; and

FIGS. 13-14 illustrate selected aspects of a stent holder of the implant delivery system of FIG. 3, where FIG. 14 is rotated 180 degrees from FIG. 13.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

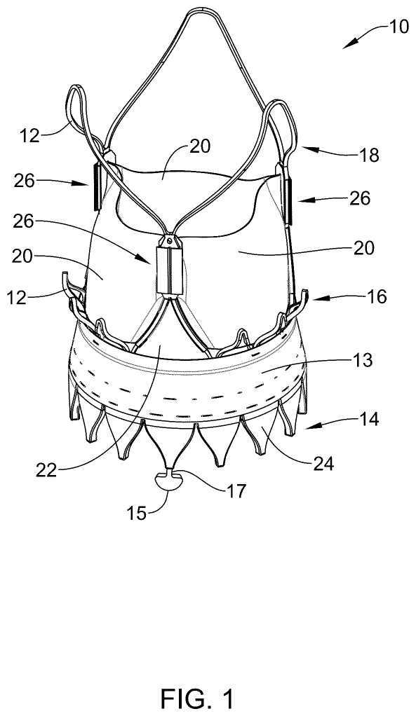

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) in a mammalian heart. The replacement heart valve implant 10 may be configured to allow one-way flow through the replacement heart valve implant 10 from an inflow end to an outflow end.

The replacement heart valve implant 10 may comprise an expandable framework 12. In some embodiments, the expandable framework 12 may have a substantially circular cross-section. In some embodiments, the expandable framework 12 may have a non-circular (e.g., D-shaped, elliptical, etc.) cross-section. In some embodiments, the expandable framework 12 may be formed from a resilient shape memory material. In at least some embodiments, the resilient shape memory material may be a metallic material such as nitinol. Some suitable but non-limiting examples of materials that may be used to form the expandable framework 12, including but not limited to metals and metal alloys, composites, polymers, and the like, are described below.

The replacement heart valve implant 10 and/or the expandable framework 12 may be configured to shift between a radially collapsed configuration and a radially expanded configuration. In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations are also contemplated. In some embodiments, the expandable framework 12 may include and/or define a plurality of interstices (e.g., openings) through the expandable framework 12.

In some embodiments, the expandable framework 12 may comprise a tubular wall 13 including a plurality of interconnected struts and defining a central lumen extending along a central longitudinal axis of the expandable framework 12. In some embodiments, the tubular wall 13 may comprise and/or define an inflow end 14 and an outflow end 16. In some embodiments, the expandable framework 12 may include a plurality of stabilization arches 18 extending downstream from the outflow end 16 and/or the tubular wall 13. In some embodiments, the outflow end 16 may be disposed longitudinally and/or axially between the inflow end 14 and the plurality of stabilization arches 18.

In some embodiments, the expandable framework 12 may comprise a plurality of commissure posts 26. In some embodiments, the plurality of commissure posts 26 may comprise three commissure posts. Other configurations, include more or fewer commissure posts than three commissure posts, are also contemplated. In at least some embodiments, the plurality of commissure posts 26 may be disposed downstream of the outflow end 16. In some embodiments, the plurality of commissure posts 26 may be disposed circumferentially around the central longitudinal axis. In some embodiments, the plurality of commissure posts 26 may be disposed longitudinally and/or axially between the outflow end 16 and the plurality of stabilization arches 18. In some embodiments, the plurality of stabilization arches 18 may extend from the plurality of commissure posts 26.

In some embodiments, the replacement heart valve implant 10 may comprise a proximal portion and a distal portion. In some embodiments, orientation of the replacement heart valve implant 10 may be related to an implant delivery system and/or a direction of implantation relative to a target site (e.g., a native heart valve). In some embodiments, the proximal portion may comprise the outflow end 16, the plurality of stabilization arches 18, and/or the plurality of commissure posts 26. In some embodiments, the distal portion may comprise the inflow end 14. Other configurations are also contemplated.

In some embodiments, the tubular wall 13 may comprise an attachment tab 15 extending upstream from the inflow end 14. The attachment tab 15 and its interaction with other components (i.e., a stent holder) are described below. In at least some embodiments, the attachment tab 15 may be aligned with a commissure post of the plurality of commissure posts 26. In some embodiments, the attachment tab 15 may be spaced apart upstream from the inflow end 14 by a neck 17 extending therebetween. In some embodiments, the attachment tab 15 and/or the neck 17 may be monolithically formed with the expandable framework 12, the tubular wall 13, and/or the plurality of interconnected struts. In some embodiments, the attachment tab 15 may extend laterally and/or circumferentially from the neck 17. In some embodiments, the attachment tab 15 may extend in opposing circumferential directions from the neck 17 (e.g., may extend both clockwise and counterclockwise from the neck 17 around the central longitudinal axis). In some alternative embodiments, the attachment tab 15 may extend in only a single direction from the neck 17 (e.g., either clockwise or counterclockwise from the neck 17 around the central longitudinal axis). In at least some embodiments, the attachment tab 15 may be substantially solid and/or devoid of apertures, spaces, gaps, or openings disposed and/or formed therein (e.g., it is not possible to place or extend any structure through the attachment tab 15).

In some embodiments, the tubular wall 13 may comprise a plurality of attachment tabs (ref. 15) extending upstream from the inflow end 14. In some embodiments, the plurality of attachment tabs (ref. 15) may be aligned with the plurality of commissure posts 26. In some embodiments, each attachment tab of the plurality of attachment tabs (ref. 15) may be aligned with one commissure post of the plurality of commissure posts 26. In some embodiments, the plurality of attachment tabs (ref. 15) may comprise three attachment tabs. Other configurations, including more or fewer attachment tabs than three attachment tabs, are also contemplated. In some embodiments, the plurality of attachment tabs (ref. 15) may be arrayed and/or circumferentially spaced apart around the central longitudinal axis. In some embodiments, the plurality of attachment tabs (ref. 15) may be arrayed and/or circumferentially spaced apart around the central longitudinal axis at equal intervals. Other configurations are also contemplated.

In some embodiments, each attachment tab of the plurality of attachment tabs (ref. 15) may be spaced apart upstream from the inflow end 14 by a neck (ref. 17). In some embodiments, each attachment tab of the plurality of attachment tabs (ref. 15) and/or its respective neck (ref. 17) may be monolithically formed with the expandable framework 12, the tubular wall 13, and/or the plurality of interconnected struts. In some embodiments, each attachment tab of the plurality of attachment tabs (ref. 15) may extend laterally and/or circumferentially from its respective neck (ref. 17). In some embodiments, each attachment tab of the plurality of attachment tabs (ref. 15) may extend in opposing circumferential directions from its respective neck (ref. 17) (e.g., may extend both clockwise and counterclockwise from its respective neck (ref. 17) around the central longitudinal axis). In some alternative embodiments, each attachment tab of the plurality of attachment tabs (ref. 15) may extend in only a single direction from its respective neck (ref. 17) (e.g., either clockwise or counterclockwise from its respective neck (ref. 17) around the central longitudinal axis). In some embodiments, each attachment tab of the plurality of attachment tabs (ref. 15) may be substantially solid and/or devoid of apertures, spaces, gaps, or openings disposed and/or formed therein (e.g., it is not possible to place or extend any structure through any of the attachment tabs).

In some embodiments, the replacement heart valve implant 10 may comprise a plurality of valve leaflets 20 disposed within the central lumen. The plurality of valve leaflets 20 may be coupled, secured, and/or fixedly attached to the expandable framework 12 and/or the plurality of commissure posts 26. One or more means of securing the plurality of valve leaflets 20 to the expandable framework 12 and/or the plurality of commissure posts 26 may be used, including but not limited to, adhesive bonding, suturing, friction fit (e.g., pinching), etc. In some embodiments, the plurality of stabilization arches 18 may extend axially away from the plurality of valve leaflets 20 and/or from the plurality of commissure posts 26 or an attachment point (or attachment points) of the plurality of valve leaflets 20 with the expandable framework 12.

In some embodiments, each valve leaflet of the plurality of valve leaflets 20 may include a root edge coupled to the expandable framework 12 and a free edge (e.g., a coaptation edge) movable relative to the root edge to coapt with the free edges of the other leaflets along a coaptation region. In some embodiments, the plurality of valve leaflets 20 may be monolithically formed with each other, such that the plurality of valve leaflets 20 is formed as a single unitary and/or monolithic unit. In some embodiments, the plurality of valve leaflets 20 may be formed monolithically with other structures such as an inner skirt 22 and/or an outer skirt 24, base structures, liners, or the like.

The plurality of valve leaflets 20 may be configured to substantially restrict fluid from flowing through the replacement heart valve implant 10 and/or the central lumen in a closed position. For example, in some embodiments, the free edges of the plurality of valve leaflets 20 may move into coaptation with one another in the closed position to substantially restrict fluid from flowing through the replacement heart valve implant 10 and/or the central lumen. The free edges of the plurality of valve leaflets 20 may be spaced apart from each other in an open position to permit fluid flow through the replacement heart valve implant 10 and/or the central lumen. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12 and/or the tubular wall 13. In at least some embodiments, the inner skirt 22 may be fixedly attached to the expandable framework 12 and/or the tubular wall 13. The inner skirt 22 may direct fluid, such as blood, flowing through the replacement heart valve implant 10 and/or the central lumen toward the plurality of valve leaflets 20. In some embodiments, the inner skirt 22 may be fixedly attached to and/or monolithically formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along the outer surface of the expandable framework 12 and/or the tubular wall 13. In some embodiments, the outer skirt 24 may be disposed at and/or adjacent the inflow end 14. The outer skirt 24 may ensure the fluid flows through the central lumen of the replacement heart valve implant 10 and does not flow around the replacement heart valve implant 10 (e.g., between the expandable framework 12 and the vessel wall), so as to ensure that the plurality of valve leaflets 20 can stop the flow of fluid when in the closed position.

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, or each of a plurality of interstices formed in the expandable framework 12 by and/or between the plurality of interconnected struts. In at least some embodiments, sealing one of, some of, or each of the plurality of interstices may be considered to prevent fluid from flowing through one of, some of, or each of the plurality of interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 23 millimeters (mm) (0.906 inches (in)), about 25 mm (0.984 in), about 27 mm (1.063 in), about 30 mm (1.181 in), etc. in an unconstrained configuration (e.g., in the deployed configuration). In some embodiments, the expandable framework 12 and/or the replacement heart valve implant 10 may have an outer extent of about 10 mm (0.394 in), about 9 mm (0.354 in) about 8 mm (0.315 in), about 7 mm (0.276 in), about 6 mm (0.236 in), etc. in the delivery configuration. Other configurations are also contemplated.

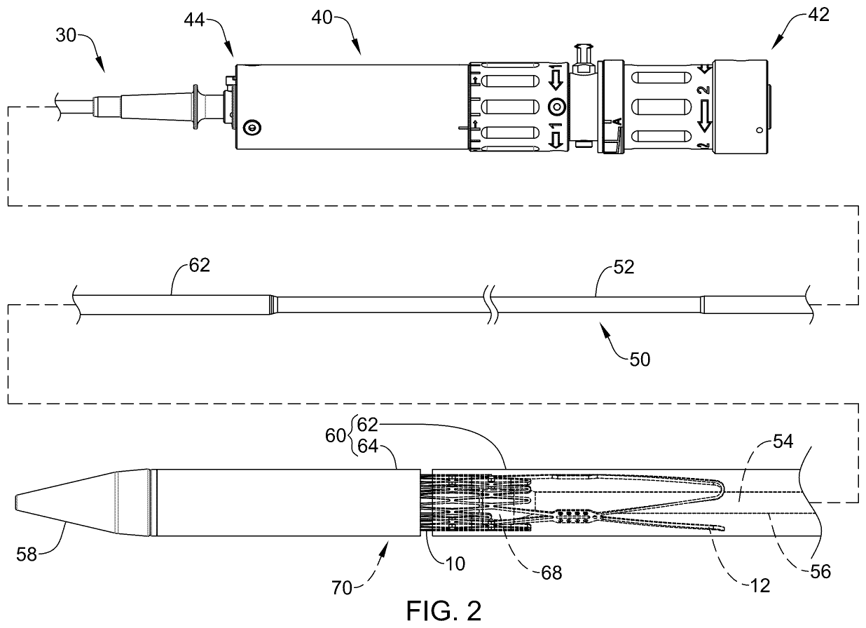

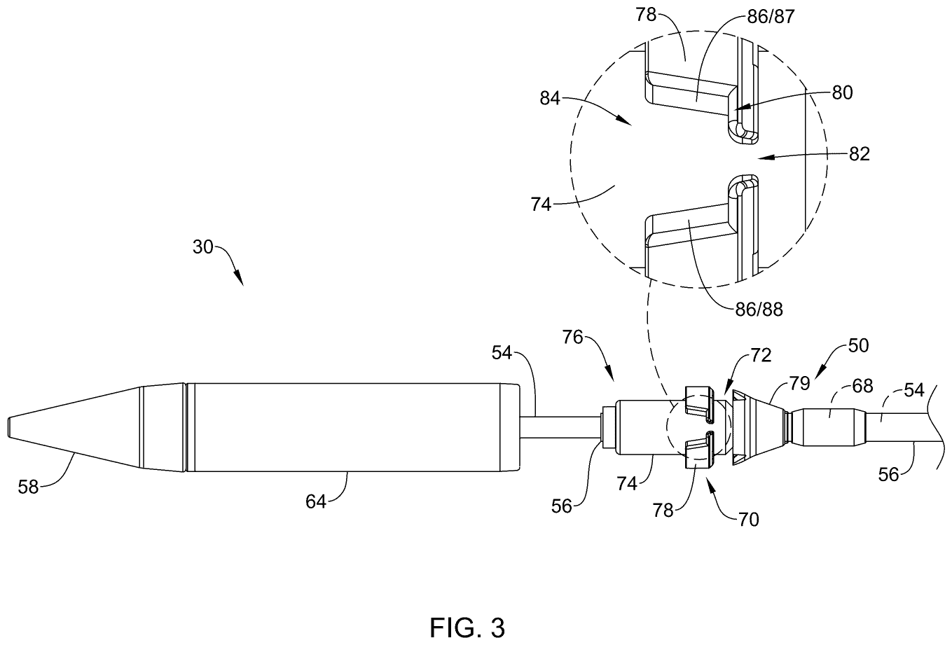

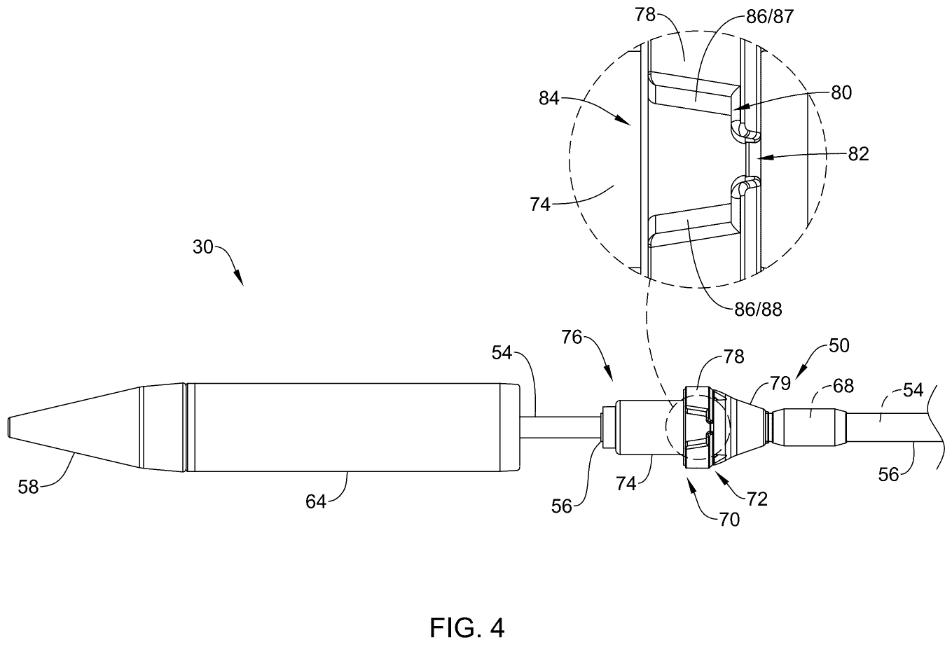

FIGS. 2-4 illustrate selected aspects of a replacement heart valve system including the replacement heart valve implant 10 and an implant delivery system 30 for delivering a replacement heart valve implant to a native heart valve (e.g., the aortic valve). The implant delivery system 30 may be compatible with and/or usable with the replacement heart valve implant 10. It should be noted that FIG. 2 includes at least one change of scale (e.g., all parts of the figure are not drawn to the same scale) to improve viewability and show additional detail of selected aspects of the implant delivery system 30. Additionally, the expandable framework 12 is shown in FIG. 2 in the radially collapsed configuration but some elements of the replacement heart valve implant 10 are not shown to improve clarity. In FIGS. 3-4, the replacement heart valve implant 10 has been omitted.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob and a second rotatable knob. In at least some embodiments, the first rotatable knob and/or the second rotatable knob may be configured to rotate around a central longitudinal axis of the implant delivery system 30 and/or the handle 40. Other configurations are also contemplated.

In some embodiments, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration (e.g., FIG. 2). The elongate shaft assembly 50 may include an outer tubular member 52 extending distally from the handle 40 and an inner shaft 54 extending distally from the handle 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and around and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle 40.

In some embodiments, the handle 40 may be configured to manipulate and/or translate the proximal sheath 62 and/or the distal sheath 64 relative to each other using the first rotatable knob and/or the second rotatable knob. In some embodiments, the handle 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle 40 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 and/or the expandable framework 12 may be disposed at least partially within the proximal sheath 62 and/or the distal sheath 64 in the radially collapsed configuration in a closed configuration of the implant holding portion 60 (e.g., FIG. 2). In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 10 and/or the expandable framework 12 in the radially collapsed configuration when the implant holding portion 60 is in the closed configuration.

In some embodiments, the implant holding portion 60 may include a stent holder 70 configured to engage the attachment tab 15 and/or the plurality of attachment tabs (ref. 15) in the radially collapsed configuration to axially secure the replacement heart valve implant 10 and/or the expandable framework 12 relative to the implant delivery system 30 and/or the stent holder 70. In at least some embodiments, the stent holder 70 may be configured to releasably engage the attachment tab 15 and/or the plurality of attachment tabs (ref. 15) in the radially collapsed configuration. In some embodiments, the stent holder 70 may be configured to releasably engage the attachment tab 15 and/or the plurality of attachment tabs (ref. 15) when the replacement heart valve implant 10 and/or the expandable framework 12 is constrained within the implant holding portion 60 of the implant delivery system 30 in the radially collapsed configuration and when the implant holding portion 60 is in the closed configuration. Additional details regarding the stent holder 70 are shown in FIGS. 3-4 and discussed below.

In some embodiments, the proximal sheath 62 may be configured to cover the proximal portion of the replacement heart valve implant 10 and/or the expandable framework 12 in the radially collapsed configuration when the implant holding portion 60 is in the closed configuration, and the distal sheath 64 may be configured to cover the distal portion of the replacement heart valve implant 10 and/or the expandable framework 12 in the radially collapsed configuration when the implant holding portion 60 is in the closed configuration. In some embodiments, the replacement heart valve implant 10 and/or the expandable framework 12 may be constrained in the radially collapsed configuration by the proximal sheath 62 and the distal sheath 64 in the closed configuration of the implant holding portion 60. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10 and/or the expandable framework 12. In some embodiments, the proximal sheath 62 may be axially spaced apart from the distal sheath 64 in the closed configuration by less than 15% of an overall length of the replacement heart valve implant 10 and/or the expandable framework 12. In some embodiments, the proximal sheath 62 may be axially spaced apart from the distal sheath 64 in the closed configuration by less than 10% of an overall length of the replacement heart valve implant 10 and/or the expandable framework 12. In some embodiments, the proximal sheath 62 may be axially spaced apart from the distal sheath 64 in the closed configuration by less than 5% of an overall length of the replacement heart valve implant 10 and/or the expandable framework 12. Other configurations are also contemplated.

In at least some embodiments, the stent holder 70 may be fixedly attached to the elongate shaft assembly 50. In some embodiments, the stent holder 70 may be fixedly attached to the intermediate tubular member 56 of the elongate shaft assembly 50. In some embodiments, the stent holder 70 may be integrally formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the implant delivery system 30.

The implant delivery system 30 and/or the elongate shaft assembly 50 may include a primary visual indicator 68 configured and/or adapted to be visible under fluoroscopy with an imaging device. Other imaging means suitable for use with transcatheter surgical procedures are also contemplated. The implant delivery system 30 and/or the primary visual indicator 68 may be configured to cooperate with the imaging device to position the replacement heart valve implant 10 at a desired insertion depth within the native heart valve (e.g., the aortic valve).

In use, the implant delivery system 30 may be advanced percutaneously through the vasculature to a position adjacent to the treatment site (e.g., the native valve annulus). For example, the implant delivery system 30 may be advanced through the vasculature and across the aortic arch to a position adjacent to the native heart valve (e.g., the aortic valve). Alternative approaches to treat a defective aortic valve and/or other heart valve(s) are also contemplated with the implant delivery system 30.

The desired insertion depth may be selected to maximize radially outward force of the expandable framework 12 within the native heart valve (e.g., the aortic valve). Positioning the replacement heart valve implant 10 at the desired insertion depth and/or within a maximum tolerance from the desired insertion depth, the replacement heart valve implant 10 and/or the expandable framework 12 may exhibit optimal arching within the native heart valve (e.g., the aortic valve) and thereby prevent migration of the replacement heart valve implant 10 and/or the expandable framework 12 downstream (or upstream).

Positioning the replacement heart valve implant 10 and/or the expandable framework 12 within the native heart valve (e.g., the aortic valve) may be accomplished by locating the primary visual indicator 68 relative to the native heart valve (e.g., the aortic valve). During visualization, the native heart valve (e.g., the aortic valve) may be identified and/or visualized under fluoroscopy using known means and/or methods, such as contrast injection.

FIGS. 3-4 illustrate selected aspects of the implant delivery system 30 of FIG. 2 in accordance with the disclosure. For improved clarity, the replacement heart valve implant 10 and some portions of the implant delivery system 30 are not shown. For example, in FIGS. 3-4, the distal sheath 64 is shown while the proximal sheath 62 is not shown.

In some embodiments, the primary visual indicator 68 may be fixedly attached to the elongate shaft assembly 50 and/or the intermediate tubular member 56. In some embodiments, the primary visual indicator 68 may be a marker band embedded within the elongate shaft assembly 50. In some embodiments, the primary visual indicator 68 may be fixedly attached to the elongate shaft assembly 50 and/or the intermediate tubular member 56 by a shrink wrap or by an adhesive element. Other configurations are also contemplated. As discussed herein, the primary visual indicator 68 may be configured and/or adapted to be visible under fluoroscopy with the imaging device. The primary visual indicator 68 may be formed from a radiopaque material. Some suitable but non-limiting examples of radiopaque materials for the primary visual indicator 68 are described below.

In some embodiments, the implant delivery system 30 and/or the elongate shaft assembly 50 may include the stent holder 70 configured to engage the expandable framework 12 of the replacement heart valve implant 10 in the radially collapsed configuration and/or when the replacement heart valve implant 10 is constrained within the implant holding portion 60 of the implant delivery system 30. In some embodiments, the stent holder 70 may comprise a body 74 extending from a first end 72 to a second end 76 disposed opposite the first end 72. In at least some embodiments, the first end 72 may be a proximal end of the stent holder 70 and/or the body 74, and the second end 76 may be a distal end of the stent holder 70 and/or the body 74.

In some embodiments, the stent holder 70 may comprise a circumferential extension 78 extending radially outward from the body 74 the stent holder 70. In some embodiments, the circumferential extension 78 may extend radially outward from a medial portion of the body 74. In some embodiments, the circumferential extension 78 may be axially spaced apart from the first end 72 and/or the second end 76, as seen in FIG. 3. In some embodiments, the circumferential extension 78 may be disposed at and/or adjacent to the first end 72, as seen in FIG. 4. Other configurations are also contemplated.

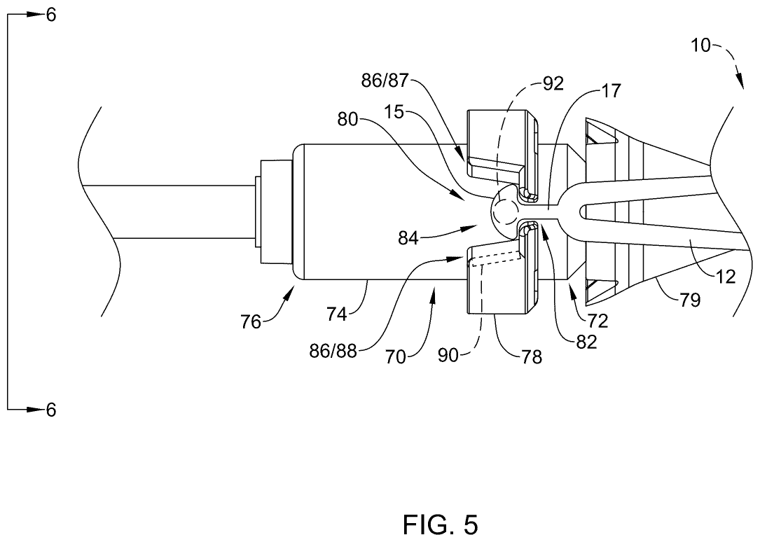

In some embodiments, the circumferential extension 78 may comprise a notch 80 configured to receive the attachment tab 15 in the radially collapsed configuration, as seen in FIGS. 5-10. Returning to FIGS. 3-4, in some embodiments, the notch 80 may have a proximal aperture 82 configured to receive the neck 17 and a distally facing recess 84 configured to receive the attachment tab 15 in the radially collapsed configuration. In some embodiments, the proximal aperture 82 may be disposed at a proximal end of the circumferential extension 78. In some embodiments, the distally facing recess 84 may extend to a distal end of the circumferential extension 78. In some embodiments, the circumferential extension 78 and/or the notch 80 may comprise a shoulder 86. In some embodiments, the circumferential extension 78 and/or the notch 80 may comprise a first shoulder 87 and a second shoulder 88 at least partially defining the distally facing recess 84. In some embodiments, the first shoulder 87 and the second shoulder 88 may extend generally longitudinally and/or in a longitudinal direction along the body 74. In some embodiments, the notch 80, the proximal aperture 82, and/or the distally facing recess 84 may open radially outward from the circumferential extension 78 and/or the body 74 of the stent holder 70. Other configurations are also contemplated. In one non-limiting alternative, the notch 80 may comprise a proximal aperture and a radially outwardly opening recess having a closed distal end.

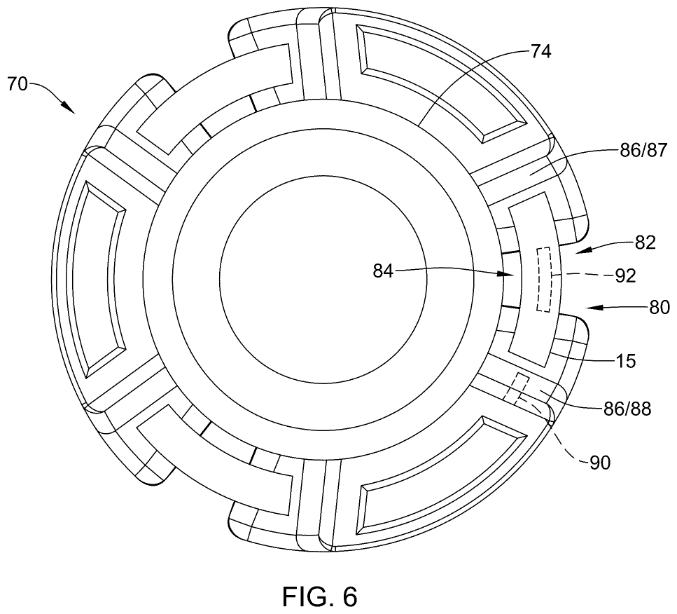

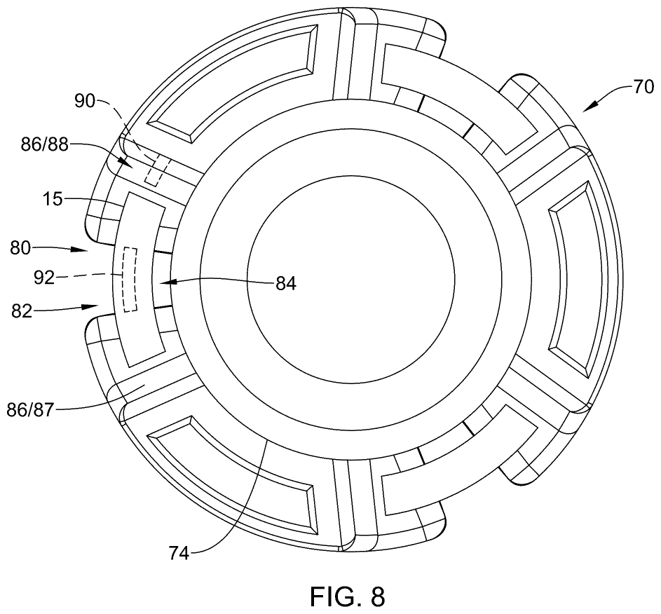

In some embodiments, the stent holder 70 and/or the circumferential extension 78 may comprise a plurality of notches (ref. 80) configured to receive a plurality of attachment tabs (ref. 15) in the radially collapsed configuration, as seen in FIGS. 6 and 8. In some embodiments, the plurality of notches (ref. 80) may comprise three notches. Other configurations, including more or fewer notches than three notches, are also contemplated. In some embodiments, the plurality of notches (ref. 80) may be arrayed and/or circumferentially spaced apart around the stent holder 70 and/or the body 74. In some embodiments, the plurality of notches (ref. 80) may be arrayed and/or circumferentially spaced apart around the stent holder 70 and/or the body 74 at equal intervals. Other configurations are also contemplated. In some embodiments, each notch (ref. 80) of the plurality of notches (ref. 80) may be formed and/or configured in accordance with the discussion above regarding the notch 80. In some embodiments, each notch (ref. 80) of the plurality of notches (ref. 80) may be configured to receive one attachment tab (ref. 15) of the plurality of attachment tabs (ref. 15) in the radially collapsed configuration.

In some embodiments, the notch 80 may be configured and/or adapted to engage the attachment tab 15 of the expandable framework 12 of the replacement heart valve implant 10 in the radially collapsed configuration and/or when the replacement heart valve implant 10 is constrained within the implant holding portion 60 of the implant delivery system 30 such that proximal axial movement of the replacement heart valve implant 10 and/or the expandable framework 12 relative to the stent holder 70 and/or the elongate shaft assembly 50 is prevented. In some embodiments, a proximal facing surface of the circumferential extension 78 may be configured and/or adapted to engage the inflow end 14 of the expandable framework 12 and/or the tubular wall 13 of the replacement heart valve implant 10 in the radially collapsed configuration and/or when the replacement heart valve implant 10 is constrained within the implant holding portion 60 of the implant delivery system 30 such that distal axial movement of the replacement heart valve implant 10 and/or the expandable framework 12 relative to the stent holder 70 and/or the elongate shaft assembly 50 is prevented. In some embodiments, the proximal aperture 82 may be configured to engage the neck 17 in the radially collapsed configuration and/or when the replacement heart valve implant 10 is constrained within the implant holding portion 60 of the implant delivery system 30 such that circumferential and/or rotational movement of the replacement heart valve implant 10 and/or the expandable framework 12 relative to the stent holder 70 and/or the elongate shaft assembly 50 is prevented.

In some embodiments, the distal sheath 64 may extend proximally from the distal tip 58. In some embodiments, the implant delivery system 30 and/or the elongate shaft assembly 50 may include a cage (not shown) disposed radially outward of and/or extending radially outward from the elongate shaft assembly 50 and/or the second end 76 of the stent holder 70. In some embodiments, the cage may be positioned radially outward of and axially overlapping the body 74 of the stent holder 70. In some embodiments, the cage may be configured to substantially center the distal sheath 64 over and/or around the elongate shaft assembly 50 and/or the replacement heart valve implant 10 (not shown) as the distal sheath 64 is moved from an open configuration to the closed configuration. Other configurations are also contemplated.

In some embodiments, the implant delivery system 30 and/or the implant holding portion 60 may include an atraumatic transition shield 79. The atraumatic transition shield 79 may be disposed adjacent the stent holder 70. In some embodiments, the atraumatic transition shield 79 may be disposed between the stent holder 70 and the handle 40. In some embodiments, the atraumatic transition shield 79 may be disposed proximal of the stent holder 70. In some embodiments, the atraumatic transition shield 79 may be disposed at and/or adjacent the first end 72 of the stent holder 70. In some embodiments, the atraumatic transition shield 79 may axially overlap the first end 72 of the stent holder 70. In some embodiments, the atraumatic transition shield 79 may be disposed radially outward of at least a portion of the first end 72 of the stent holder 70. In some embodiments, the atraumatic transition shield 79 may be tapered radially inward in the proximal direction and/or toward the handle 40. The atraumatic transition shield 79 may be configured to prevent the replacement heart valve implant 10, the expandable framework 12, the plurality of valve leaflets 20, etc. (not shown in all figures) from catching on the stent holder 70 as the implant delivery system 30 is withdrawn after deploying the replacement heart valve implant 10.

In use, after advancing and/or navigating the implant delivery system 30 and/or the implant holding portion 60 to the treatment site (over a guidewire, for example), the proximal sheath 62 and/or the distal sheath 64 may be axially translated relative to each other to shift the implant holding portion 60 to the open configuration. When unconstrained by the implant holding portion 60, the replacement heart valve implant 10 (e.g., FIG. 1) and/or the expandable framework 12 (e.g., FIG. 1) may be configured to shift from the radially collapsed configuration (e.g., FIG. 2) to the radially expanded configuration (e.g., FIG. 1). Shifting the replacement heart valve implant 10 (e.g., FIG. 1) and/or the expandable framework 12 toward the radially expanded configuration after axially translating the proximal sheath 62 and/or the distal sheath 64 away from each other may permit the replacement heart valve implant 10 (e.g., FIG. 1) and/or the expandable framework 12 to decouple and/or detach from the implant delivery system 30. Some suitable but non-limiting materials for the implant delivery system 30, the handle 40, the elongate shaft assembly 50, and/or components or elements thereof, for example metallic materials and/or polymeric materials, are described below.

During placement of the replacement heart valve implant 10 within the native heart valve (e.g., the aortic valve), fluoroscopy may be used. A common and/or standard practice involves positioning an imaging device in a 3-cusp view of the native heart valve (e.g., the aortic valve), which view is known to and understood by the skilled artisan and thus is not discussed in detail herein. It is desirable to position the plurality of commissure posts 26 of the replacement heart valve implant 10 in alignment with the commissures of the native heart valve (e.g., the aortic valve) to avoid obstruction of the coronary arteries. In the 3-cusp view, which roughly correlates to a side view of the replacement heart valve implant 10 within the native heart valve (e.g., the aortic valve), the plurality of commissure posts 26 may be visible (at least partially) under fluoroscopy. In the 3-cusp view, one commissure post of the plurality of commissure posts 26 may be disposed in a middle position between the other two commissure posts. However, in some instances, the 3-cusp view may be inconclusive regarding the rotational orientation of the expandable framework 12 and/or the replacement heart valve implant 10 relative to the native heart valve (e.g., the aortic valve). For example, it may be unclear whether the middle commissure post is disposed in an anterior position or a posterior position with respect to the imaging device. Positioning the middle commissure post in the posterior position is the correct rotation orientation to align with the commissures of the native heart valve (e.g., the aortic valve), while positioning the middle commissure post in the anterior position may result in misalignment of the plurality of commissure posts 26 with the commissures of the native heart valve and/or coronary artery obstruction. In some instances, secondary views using the imaging device are necessary to determine the actual rotational orientation of the expandable framework 12 and/or the replacement heart valve implant 10 relative to the native heart valve (e.g., the aortic valve). The current disclosure seeks to identify features and/or methods to determine the actual rotational orientation of the expandable framework 12 and/or the replacement heart valve implant 10 relative to the native heart valve (e.g., the aortic valve) without the use secondary views and/or changing the position of the imaging device during the procedure, thereby streamlining the procedure, reducing risk to the patient, and improving procedure results.

In some embodiments, the stent holder 70 may be configured and/or adapted to be substantially transparent under fluoroscopy. In some embodiments, the stent holder 70 may be formed from a non-radiopaque material, such as a polymer or a polymer composite. Some suitable but non-limiting materials for the stent holder 70 are described below.

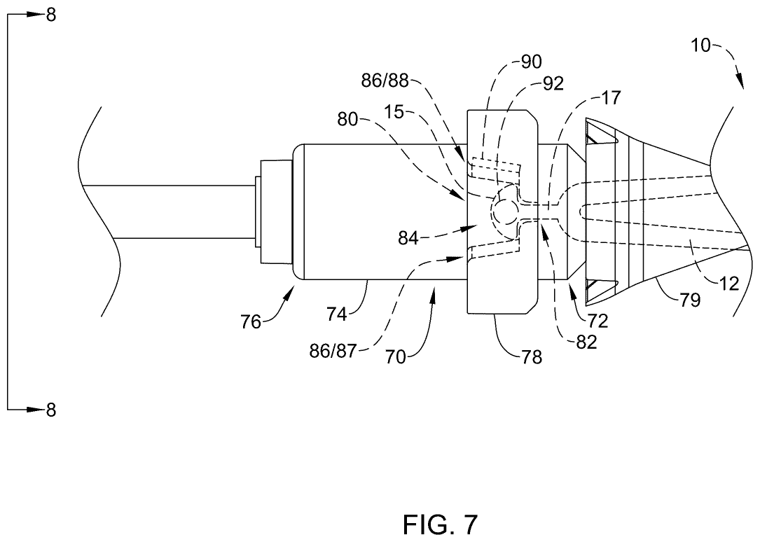

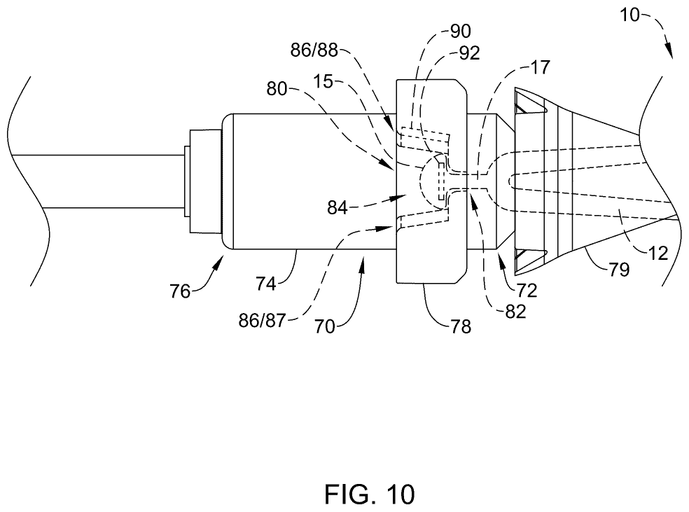

In some embodiments, the stent holder 70 may comprise a first radiopaque marker 90 and the attachment tab 15 of expandable framework 12 may comprise a second radiopaque marker 92, as seen in FIGS. 5-10. It should be noted that in FIGS. 7 and 10 some forward-facing features of the stent holder 70 have been omitted to improve clarity. The first radiopaque marker 90 may be formed from a radiopaque material. The second radiopaque marker 92 may be formed from a radiopaque material. In some embodiments, the first radiopaque marker 90 and the second radiopaque marker 92 may be formed from the same radiopaque material. In some embodiments, the first radiopaque marker 90 and the second radiopaque marker 92 may be formed from different radiopaque materials. Some suitable but non-limiting examples of radiopaque materials for the first radiopaque marker 90 and/or the second radiopaque marker 92 are discussed below.

In at least some embodiments, the first radiopaque marker 90 and the second radiopaque marker 92 may collectively indicate a rotational orientation of the expandable framework 12 and/or the replacement heart valve implant 10 relative to the native heart valve (e.g., the aortic valve) under fluoroscopy in the 3-cusp view of the native heart valve (e.g., the aortic valve) using the imaging device. In some embodiments, the first radiopaque marker 90 and the second radiopaque marker 92 may collectively indicate a correct rotational orientation of the expandable framework 12 and/or the replacement heart valve implant 10 relative to the native heart valve (e.g., the aortic valve) under fluoroscopy in the 3-cusp view of the native heart valve (e.g., the aortic valve) using the imaging device when the middle commissure post is disposed in the posterior position. In some embodiments, the first radiopaque marker 90 and the second radiopaque marker 92 may collectively indicate an incorrect rotational orientation of the expandable framework 12 and/or the replacement heart valve implant 10 relative to the native heart valve (e.g., the aortic valve) under fluoroscopy in the 3-cusp view of the native heart valve (e.g., the aortic valve) using the imaging device when the middle commissure post is disposed in the anterior position.

In some embodiments, under fluoroscopy in the 3-cusp view a first relative positioning of the first radiopaque marker 90 and the second radiopaque marker 92 may collectively indicate a correct rotational orientation of the expandable framework 12 and/or the replacement heart valve implant 10 relative to the native heart valve (e.g., the aortic valve) when the middle commissure post is disposed in the posterior position. In some embodiments, under fluoroscopy in the 3-cusp view a second relative positioning of the first radiopaque marker 90 and the second radiopaque marker 92 may collectively indicate an incorrect rotational orientation of the expandable framework 12 and/or the replacement heart valve implant 10 relative to the native heart valve (e.g., the aortic valve) when the middle commissure post is disposed in the anterior position.

In some embodiments, the first radiopaque marker 90 may extend and/or may be oriented in a generally longitudinal direction. In some embodiments, the first radiopaque marker 90 may extend and/or may be oriented in a generally longitudinal direction in the 3-cusp view under fluoroscopy. In some embodiments, the first radiopaque marker 90 may be disposed on, fixedly attached to, or disposed within the shoulder 86 (e.g., on, fixedly attached to, or disposed within the first shoulder 87, on, fixedly attached to, or disposed within the second shoulder 88, etc.) of the notch 80 and/or the circumferential extension 78. In some embodiments, the first radiopaque marker 90 may be embedded within the shoulder 86 (e.g., embedded within the first shoulder 87, embedded within the second shoulder 88, etc.) of the notch 80 and/or the circumferential extension 78. In some embodiments, the first radiopaque marker 90 may only be visible under fluoroscopy. Other configurations are also contemplated.

In some embodiments, the second radiopaque marker 92 may be disposed on, fixedly attached to, or disposed within the attachment tab 15. In some embodiments, the second radiopaque marker 92 may be disposed within a recess formed in the attachment tab 15. Other configurations are also contemplated. In some embodiments, while the expandable framework 12 and/or the attachment tab 15 may be formed from a material that may be at least partially radiopaque, the second radiopaque marker 92 may be formed from a material that is different from the expandable framework 12 and/or the attachment tab 15 such that the second radiopaque marker 92 is distinguishable from the expandable framework 12 and/or the attachment tab 15. For example, the second radiopaque marker 92 may be formed from a material that is more radiopaque than the expandable framework 12 and/or the attachment tab 15. In some embodiments, the second radiopaque marker 92 may extend laterally and/or circumferentially with respect to the central longitudinal axis of the expandable framework 12 and/or the replacement heart valve implant 10 and/or the central longitudinal axis of the implant delivery system 30.

In some embodiments, the first radiopaque marker 90 and the second radiopaque marker 92 may collectively form a symbol visible under fluoroscopy in the 3-cusp view. In some embodiments, the symbol may have a first appearance under fluoroscopy in the 3-cusp view when the expandable framework 12 is in the correct rotational orientation (e.g., when the middle commissure post is disposed in the posterior position) relative to the native heart valve (e.g., the aortic valve), as seen in FIGS. 7-8 and 10. In some embodiments, the symbol may have a second appearance under fluoroscopy in the 3-cusp view when the expandable framework 12 is in the incorrect rotational orientation (e.g., when the middle commissure post is disposed in the anterior position) relative to the native heart valve (e.g., the aortic valve), as seen in FIGS. 5-6 and 9. FIGS. 5, 7, and 9-10 are generally shown from the direction used in the 3-cusp view (looking at/into the page), although rotation of page/view may differ depending on positioning of the imaging device.

As may be seen in the figures, the second appearance of the symbol is different from the first appearance of the symbol. In at least some embodiments, the second appearance of the symbol may be a mirror image of the first appearance of the symbol. In some embodiments, the mirror image may be relative to and/or taken with respect to a plane having the central longitudinal axis of the expandable framework 12 and/or the replacement heart valve implant 10 and/or the central longitudinal axis of the implant delivery system 30 therein, wherein the plane also extends through the neck 17 coupling and/or fixing the attachment tab 15 to the expandable framework 12 (e.g., horizontal and perpendicular to the drawing sheets).

In some embodiments, the symbol may comprise a longitudinal component and a lateral component (or a circumferential component). In some embodiments, the first radiopaque marker 90 may form and/or may be the longitudinal component of the symbol. In some embodiments, the second radiopaque marker 92 may form and/or may be the lateral component (or the circumferential component) of the symbol. In some embodiments, under fluoroscopy in the 3-cusp view the lateral component of the symbol may extend in a first direction from the longitudinal component of the symbol when the expandable framework 12 is in the correct rotational orientation (e.g., when the middle commissure post is disposed in the posterior position) relative to the native heart valve (e.g., the aortic valve). In some embodiments, under fluoroscopy in the 3-cusp view the lateral component of the symbol may extend in a second direction opposite the first direction from the longitudinal component of the symbol when the expandable framework 12 is in the incorrect rotational orientation (e.g., when the middle commissure post is disposed in the anterior position) relative to the native heart valve (e.g., the aortic valve). The first and second directions may be known relative to the correct and incorrect rotational orientations prior to beginning the procedure such that they may be properly identified under fluoroscopy in the 3-cusp view during the procedure.

In some embodiments, under fluoroscopy in the 3-cusp view the symbol may form a first letter or a first recognizable character when the expandable framework 12 is in the correct rotational orientation (e.g., when the middle commissure post is disposed in the posterior position) relative to the native heart valve (e.g., the aortic valve). In some embodiments, under fluoroscopy in the 3-cusp view the symbol may form a second letter or a second recognizable character when the expandable framework 12 is in the incorrect rotational orientation (e.g., when the middle commissure post is disposed in the anterior position) relative to the native heart valve (e.g., the aortic valve). In at least some embodiments, the second letter or the second recognizable character may be a mirror image of the first letter or the first recognizable character.

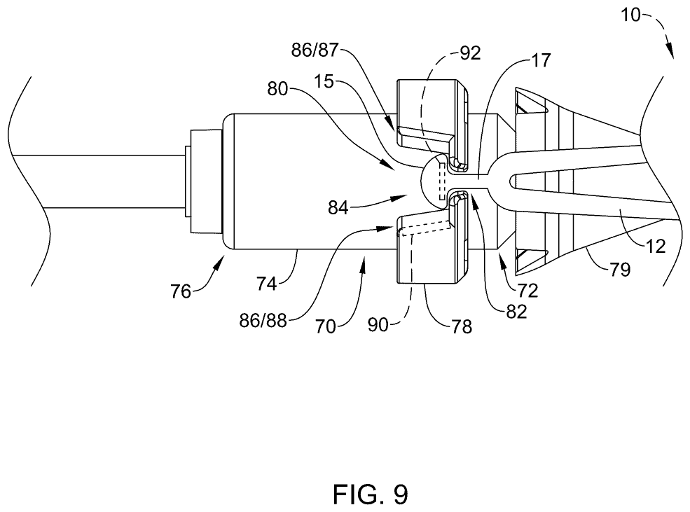

In one non-limiting example, the first letter or the first recognizable character may be and/or resemble the letter “b” and the second letter or the second recognizable character may be and/or resemble the letter “d”, as seen in FIGS. 5 and 7, respectively. Alternatively, in a second non-limiting example, the first letter or the first recognizable character may be and/or resemble the letter “d” and the second letter or the second recognizable character may be and/or resemble the letter “b”. In a third non-limiting example, the first letter or the first recognizable character may be and/or resemble the letter “L” and the second letter or the second recognizable character may be and/or resemble the letter “J”, as seen in FIGS. 9 and 10, respectively. Alternatively, in a fourth non-limiting example, the first letter or the first recognizable character may be and/or resemble the letter “J” and the second letter or the second recognizable character may be and/or resemble the letter “L”. Other configurations, letters, recognizable characters, etc. are also contemplated.

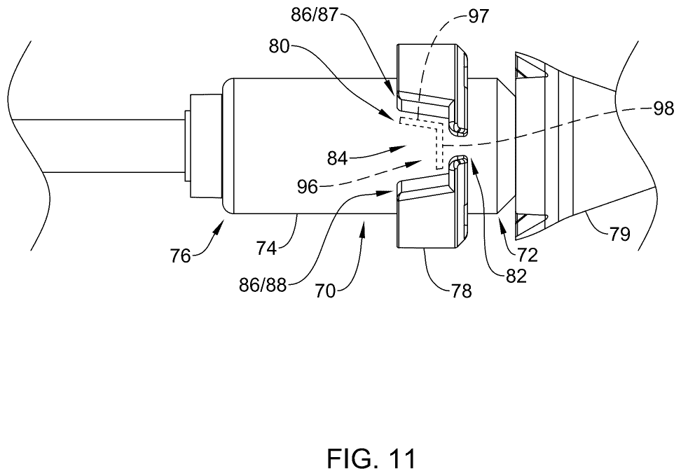

FIGS. 11-14 illustrate selected aspects of one or more alternative embodiments and/or examples of the stent holder 70, which may be formed and/or configured in accordance with the current disclosure. To improve clarity, some features are not shown in FIGS. 11-14, but may be seen in other figures. Features discussed herein but not explicitly shown may be understood from other figures. For example, the replacement heart valve implant 10 and various elements thereof are not shown in FIGS. 11-14.

In contrast to embodiments described above, in some embodiments, the stent holder 70 may comprise a first radiopaque marker 96 disposed on, fixedly attached to, or disposed within the body 74 of the stent holder 70. In some embodiments, the first radiopaque marker 96 may be embedded within the stent holder 70 and/or the body 74 of the stent holder 70. In some embodiments, the first radiopaque marker 96 may be disposed on, fixedly attached to, disposed within, or embedded within the body 74 of the stent holder 70 radially inward from the notch 80 and/or the distally facing recess 84. In some embodiments, the first radiopaque marker 96 may be circumferentially aligned with the notch 80 and/or the distally facing recess 84.

In at least some embodiments, the first radiopaque marker 96 may indicate a rotational orientation of the expandable framework 12 and/or the replacement heart valve implant 10 relative to the native heart valve (e.g., the aortic valve) under fluoroscopy in the 3-cusp view of the native heart valve (e.g., the aortic valve) using the imaging device. In some embodiments, the first radiopaque marker 96 may indicate a correct rotational orientation of the expandable framework 12 and/or the replacement heart valve implant 10 relative to the native heart valve (e.g., the aortic valve) using the imaging device when the middle commissure post of the plurality of commissure posts 26 of the expandable framework 12 is disposed in the posterior position relative to the native heart valve (e.g., the aortic valve) under fluoroscopy in the 3-cusp view of the native heart valve (e.g., the aortic valve). In some embodiments, the first radiopaque marker 96 may indicate an incorrect rotational orientation of the expandable framework 12 and/or the replacement heart valve implant 10 relative to the native heart valve (e.g., the aortic valve) using the imaging device when the middle commissure post of the plurality of commissure posts 26 of the expandable framework 12 is disposed in the anterior position relative to the native heart valve (e.g., the aortic valve) under fluoroscopy in the 3-cusp view of the native heart valve (e.g., the aortic valve).

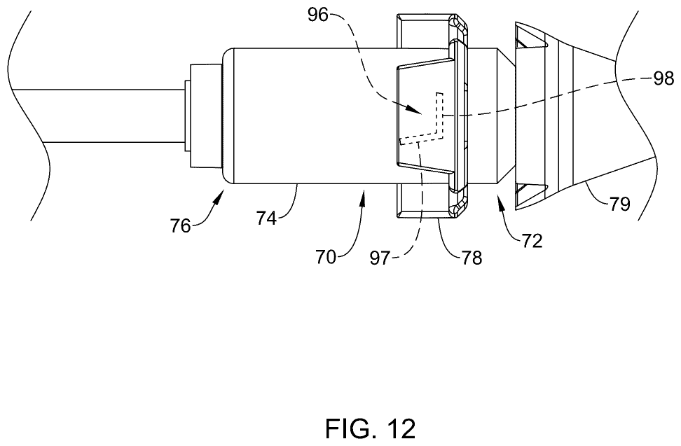

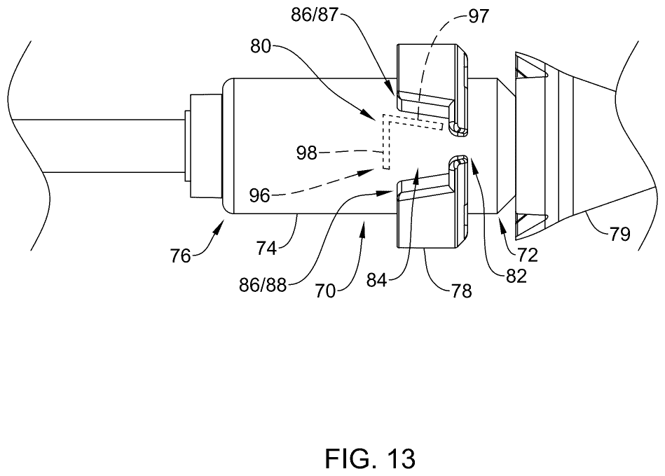

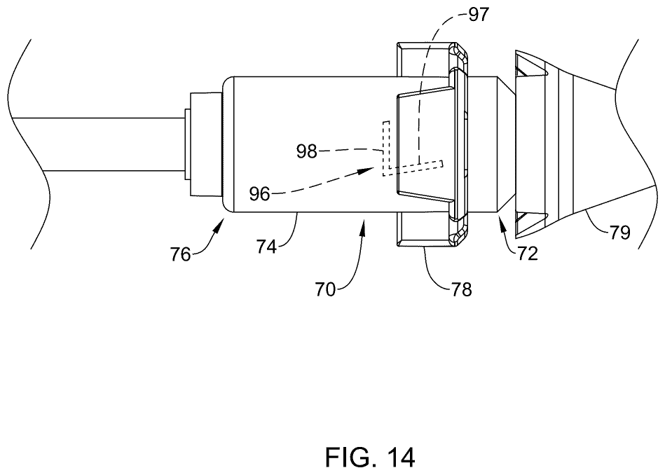

In some embodiments, the first radiopaque marker 96 may have a first appearance under fluoroscopy under fluoroscopy in the 3-cusp view when the expandable framework 12 is in the correct rotational orientation (e.g., when the middle commissure post is disposed in the posterior position) relative to the native heart valve (e.g., the aortic valve), as seen in FIGS. 12 and 14. In some embodiments, the first radiopaque marker 96 may have a second appearance under fluoroscopy in the 3-cusp view when the expandable framework 12 is in the incorrect rotational orientation (e.g., when the middle commissure post is disposed in the anterior position) relative to the native heart valve (e.g., the aortic valve), as seen in FIGS. 11 and 13. FIGS. 11-14 are generally shown from the direction used in the 3-cusp view (looking at/into the page), although rotation of page/view may differ depending on positioning of the imaging device.

As may be seen in the figures, the second appearance of the first radiopaque marker 96 may be different from the first appearance of the first radiopaque marker 96. In at least some embodiments, the second appearance of the first radiopaque marker 96 may be a mirror image of the first appearance of the first radiopaque marker 96. In some embodiments, the mirror image may be relative to and/or taken with respect to a plane having the central longitudinal axis of the expandable framework 12 and/or the replacement heart valve implant 10 and/or the central longitudinal axis of the implant delivery system 30 therein, wherein the plane also extends through the neck 17 coupling and/or fixing the attachment tab 15 to the expandable framework 12 (e.g., horizontal and perpendicular to the drawing sheets).

In some embodiments, the first radiopaque marker 96 may comprise a longitudinal component 97 and a lateral component 98 (or a circumferential component). In some embodiments, under fluoroscopy in the 3-cusp view the lateral component 98 of the first radiopaque marker 96 may extend in a first direction from the longitudinal component 97 of the first radiopaque marker 96 when the expandable framework 12 is in the correct rotational orientation (e.g., when the middle commissure post is disposed in the posterior position) relative to the native heart valve (e.g., the aortic valve). In some embodiments, under fluoroscopy in the 3-cusp view the lateral component 98 of the first radiopaque marker 96 may extend in a second direction opposite the first direction from the longitudinal component 97 of the first radiopaque marker 96 when the expandable framework 12 is in the incorrect rotational orientation (e.g., when the middle commissure post is disposed in the anterior position) relative to the native heart valve (e.g., the aortic valve). The first and second directions may be known relative to the correct and incorrect rotational orientations prior to beginning the procedure such that they may be properly identified under fluoroscopy in the 3-cusp view during the procedure, and in some embodiments, the first and second directions may be reversed from those shown in FIGS. 11-14. Other configurations are also contemplated.

In at least some interventions, the replacement heart valve implant 10 (e.g., FIG. 1) may be deployed within the native heart valve (e.g., the native heart valve is left in place). Alternatively, the replacement heart valve implant 10 (e.g., FIG. 1) may be deployed within a previously replaced native heart valve (such as via a surgical aortic valve replacement procedure or via a transcatheter aortic valve replacement procedure) and the replacement heart valve implant 10 may be deployed within the earlier-placed implant.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the implant delivery system, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten and alloys thereof, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve system, comprising:
a replacement heart valve implant comprising an expandable framework configured to shift between a radially collapsed configuration and a radially expanded configuration, and a plurality of valve leaflets secured to the expandable framework;
wherein the expandable framework comprises a tubular wall defining an inflow end and an attachment tab extending upstream from the inflow end; and
an implant delivery system comprising a handle and an elongate shaft assembly extending distally from the handle, wherein a distal portion of the elongate shaft assembly comprises an implant holding portion including a stent holder configured to engage the attachment tab in the radially collapsed configuration to axially secure the replacement heart valve implant relative to the implant delivery system;
wherein the stent holder comprises a first radiopaque marker and the attachment tab comprises a second radiopaque marker, the first radiopaque marker and the second radiopaque marker collectively indicating a rotational orientation of the expandable framework relative to a native heart valve under fluoroscopy.

2. The replacement heart valve system of claim 1, wherein the stent holder is substantially transparent under fluoroscopy.

3. The replacement heart valve system of claim 1, wherein the stent holder comprises a notch configured to receive the attachment tab in the radially collapsed configuration.

4. The replacement heart valve system of claim 3, wherein the first radiopaque marker is disposed on or within a shoulder of the notch.

5. The replacement heart valve system of claim 1, wherein the first radiopaque marker extends in a longitudinal direction.

6. The replacement heart valve system of claim 1, wherein the first radiopaque marker and the second radiopaque marker collectively form a symbol visible under fluoroscopy.

7. The replacement heart valve system of claim 6, wherein the symbol has a first appearance under fluoroscopy when the expandable framework is in a correct rotational orientation relative to the native heart valve, and a second appearance under fluoroscopy when the expandable framework is in an incorrect rotational orientation relative to the native heart valve.

8. The replacement heart valve system of claim 7, wherein the second appearance is a mirror image of the first appearance.

9. The replacement heart valve system of claim 6, wherein the symbol comprises a longitudinal component and a lateral component.

10. The replacement heart valve system of claim 9, wherein under fluoroscopy the lateral component extends in a first direction from the longitudinal component when the expandable framework is in a correct rotational orientation relative to the native heart valve, and the lateral component extends in a second direction opposite the first direction from the longitudinal component when the expandable framework is in an incorrect rotational orientation relative to the native heart valve.

11. A replacement heart valve system, comprising:
a replacement heart valve implant comprising an expandable framework configured to shift between a radially collapsed configuration and a radially expanded configuration, and a plurality of valve leaflets secured to the expandable framework;
wherein the expandable framework comprises a tubular wall defining an inflow end and an attachment tab spaced apart upstream from the inflow end by a neck extending therebetween; and
an implant delivery system comprising a handle and an elongate shaft assembly extending distally from the handle, wherein a distal portion of the elongate shaft assembly comprises an implant holding portion including a stent holder configured to engage the attachment tab in the radially collapsed configuration to axially secure the replacement heart valve implant relative to the implant delivery system;
wherein the stent holder comprises a circumferential extension extending radially outward from a body of the stent holder;
wherein the circumferential extension comprises a notch having a proximal aperture configured to receive the neck and a distally facing recess configured to receive the attachment tab;
wherein the stent holder comprises a first radiopaque marker and the attachment tab comprises a second radiopaque marker, the first radiopaque marker and the second radiopaque marker collectively indicating a rotational orientation of the expandable framework relative to a native heart valve under fluoroscopy.

12. The replacement heart valve system of claim 11, wherein the proximal aperture and the distally facing recess both open radially outward from the circumferential extension.

13. The replacement heart valve system of claim 11, wherein under fluoroscopy a first relative positioning of the first radiopaque marker and the second radiopaque marker collectively indicates a correct rotational orientation of the expandable framework relative to the native heart valve, and a second relative positioning of the first radiopaque marker and the second radiopaque marker collectively indicates an incorrect rotational orientation of the expandable framework relative to the native heart valve.

14. The replacement heart valve system of claim 11, wherein the implant holding portion comprises a distal sheath and a proximal sheath configured to constrain the replacement heart valve implant in the radially collapsed configuration with the attachment tab engaged with the stent holder.

15. A replacement heart valve system, comprising:
a replacement heart valve implant comprising an expandable framework configured to shift between a radially collapsed configuration and a radially expanded configuration, and a plurality of valve leaflets secured to the expandable framework;
wherein the expandable framework comprises a tubular wall defining an inflow end and an attachment tab extending upstream from the inflow end; and
an implant delivery system comprising a handle and an elongate shaft assembly extending distally from the handle, wherein a distal portion of the elongate shaft assembly comprises an implant holding portion including a stent holder configured to engage the attachment tab in the radially collapsed configuration to axially secure the replacement heart valve implant relative to the implant delivery system;
wherein the stent holder is substantially transparent under fluoroscopy;
wherein the stent holder comprises a first radiopaque marker embedded therein, the first radiopaque marker indicating when a middle commissure post of the expandable framework is disposed in a posterior position and when the middle commissure post of the expandable framework is disposed in an anterior position relative to a native heart valve under fluoroscopy in a 3-cusp view.

16. The replacement heart valve system of claim 15, wherein the first radiopaque marker has a first appearance under fluoroscopy in the 3-cusp view when the middle commissure post of the expandable framework is disposed in the posterior position, and a second appearance under fluoroscopy in the 3-cusp view when the middle commissure post of the expandable framework is disposed in the anterior position.

17. The replacement heart valve system of claim 16, wherein the second appearance is a mirror image of the first appearance.

18. The replacement heart valve system of claim 15, wherein the first radiopaque marker comprises a longitudinal component and a lateral component.

19. The replacement heart valve system of claim 18, wherein under fluoroscopy in the 3-cusp view, the lateral component extends in a first direction from the longitudinal component when the middle commissure post of the expandable framework is disposed in the posterior position, and the lateral component extends in a second direction opposite the first direction from the longitudinal component when the middle commissure post of the expandable framework is disposed in the anterior position.

20. The replacement heart valve system of claim 15, wherein the stent holder comprises a circumferential extension extending radially outward from a body of the stent holder, the circumferential extension comprising a notch configured to receive the attachment tab in the radially collapsed configuration;
wherein the first radiopaque marker is embedded within the body of the stent holder radially inward from and circumferentially aligned with the notch.
